(19) United States
(12) Patent Application Publication
(10) Pub. No.: US 2012/0010656 A1
(43) Pub. Date: Jan. 12, 2012

(54) Suture Device with Suture Thread of Varying Diameters

(76) Inventor: Hsieh-Hsing Lin, New Taipei City (TW)

(21) Appl. No.: 13/174,843

(22) Filed: Jul. 1, 2011

(30) Foreign Application Priority Data
Jul. 9, 2010 (TW) 099213145

(51) Int. Cl. A61B 17/06

(52) U.S. Cl. 606/224

(57) ABSTRACT

The suture device in accordance with the present invention has a needle and a suture thread. The needle has a point formed at a first end of the needle. The suture thread is connected to the needle and has a first section and a second section. The first section has an end connected to a second end of the needle. The second section has a diameter different from a diameter of the first section and an end connected to an opposite end of the first section.

### BACKGROUND OF THE INVENTION

1. Field of the Invention

The present invention relates to a suture device, especially to a surgical suture device.

2. Description of the Prior Art

After injury or surgery, conventional suture devices are used for suturing closure of wounds or surgical incisions. The closing process generally includes two steps. In the first step of the closing process, a wound or incision is preliminarily sutured with a first suture device that employees a thicker suture thread. In the second step of the closing process, the wound or incision is then precisely sutured with a second suture device that employees a thinner suture thread.

In other words, it requires two or more than two conventional suturing devices to close a wound or an incision. Each of a conventional suture device comprises a needle and a suture thread connected to the needle. A conventional suture device used for preliminary suturing comprises thicker suture thread while another conventional suture device used for fine or precise suturing comprises thinner suture thread.

However, with the at least two conventional suture devices required to close a wound or an incision, a physician or a surgeon will have to spend more time handling multiple suture devices to carry out the closing process, which is significantly inconvenient.

To overcome the shortcomings, the present invention provides a surgical suture device with a suture thread of varying diameters to mitigate or obviate the aforementioned problems.

### SUMMARY OF THE INVENTION

The main objective of the invention is to provide a suture device with a suture thread of varying diameters.

The suture device in accordance with the present invention has a needle and a suture thread. The needle has a point formed at a first end of the needle. The suture thread is connected to the needle and comprises a first section and a second section. The first section comprises an end connected to a second end of the needle. The second section comprises a diameter different from a diameter of the first section and an end connected to an opposite end of the first section.

The single suture thread of the suture device of the present invention has varying diameters. Specifically, the suture thread comprises a first section connected to the needle and other section or sections sequentially connected to the first section, wherein the other section or sections have a diameter or diameters different from the diameter of the first section. The suture device in accordance with the present invention enables a physician or a surgeon to perform both the preliminary suturing and precise suturing required in a closure process and thus shortens operation time and provides great convenience.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of the first embodiment of the suture device with a suture thread of varying diameters in accordance with the present invention;

FIG. 2 is an operational top view of the suture device in FIG. 1 used in closing an incision;

FIG. 3 is a perspective view of the second embodiment of the suture device with a suture thread of varying diameters in accordance with the present invention; and

FIG. 4 is an operational top view of the suture device in FIG. 3 used in closing an U-shaped incision.

### DETAILED DESCRIPTION OF THE PREFERRED EMBODIMENTS

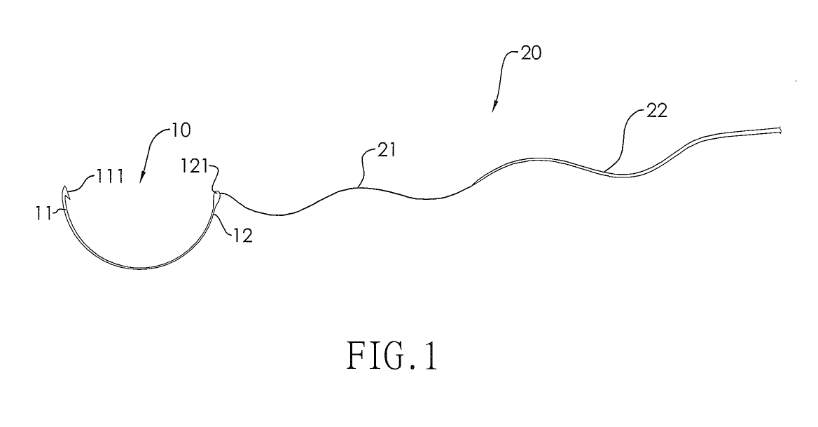

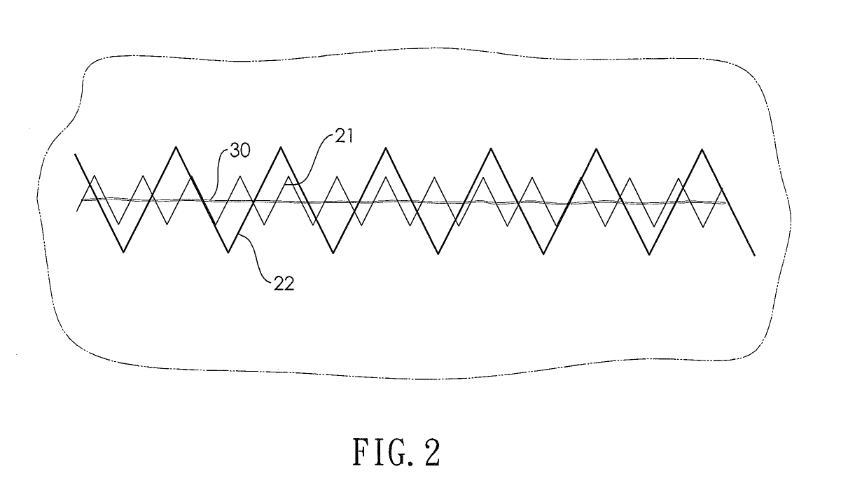

With reference to FIGS. 1 and 2, the first embodiment of the suture device with a suture thread of varying diameters in accordance with the present invention comprises a needle 10 and a suture thread 20. The needle 10 may be made from stainless steel. The suture thread 20 may be made from nylon or other fabric fibers.

The needle 10 comprises a first end 11, a second end 12 opposite to each other, and a point 111 formed at the first end. In the instant embodiment, the needle 10 is curved and the needle 10 comprises a connection portion 121 formed at the second end of the needle 10.

The suture thread 20 comprises an end connected to the second end 12 of the needle 10. The suture thread 20 comprises a first section 21 and a second section 22. The first section 21 has a diameter smaller than a diameter of the second section 22. The first section 21 comprises an end connected to the second end 12 of the needle 10. The second section 22 has an end connected to an opposite end of the first section 21. In the instant embodiment, the first section 21 of the suture thread 20 is tied to the connecting portion 121 of the second end 12 of the needle 10.

With the foregoing structure, when performing a closure process on a surgical incision 30, the surgeon firstly inserts the needle 10 into the neighboring tissue and pulls the suture thread 20 that the second section 22 corresponds to the incision 30, so as to perform a preliminary suturing for the incision 30. Second, the surgeon may then pull the suture thread 20 so to correspond the first section 21 to the incision 30 to perform a precise suturing. A first-section-side portion of the second section 22 left unused and exposed outside the body of the patient may be removed later, for example, at the completion of the closure process.

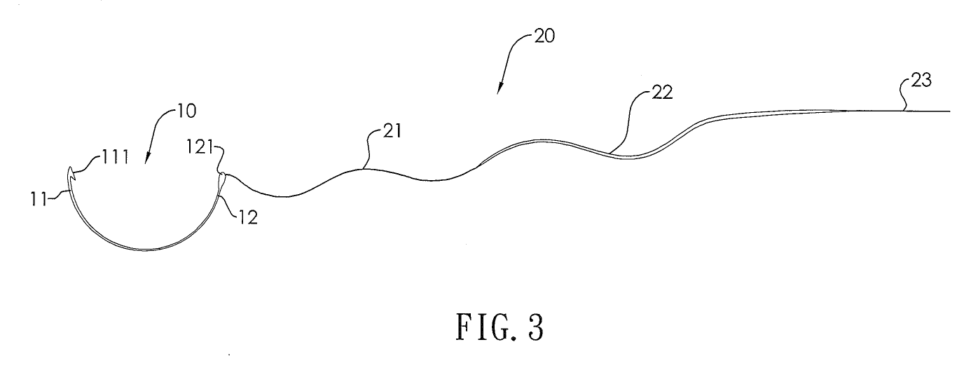

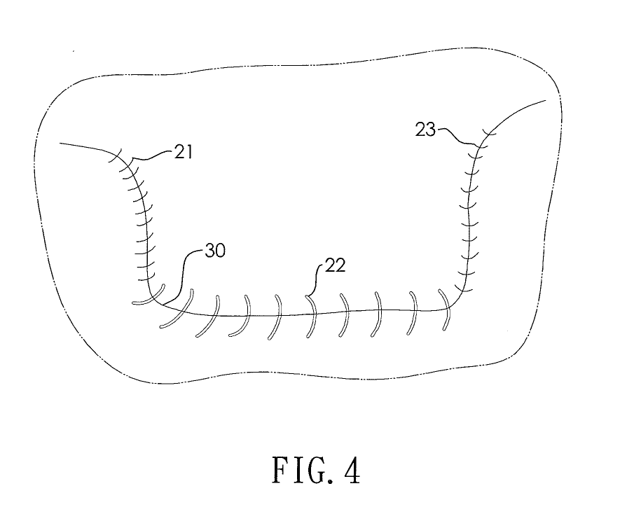

With reference to FIGS. 3 and 4, the second embodiment of the suture device with a suture thread of varying diameters in accordance with the present invention comprises a needle 10 and a suture thread 20. The needle 10 has a similar structure as the first embodiment and thus redundant descriptions there for is omitted herein.

The suture thread 20 comprises an end connected to the second end 12 of the needle 10. The suture thread 20 comprises a first section 21, a second section 22 and a third section 23. The first section 21 has a diameter smaller than a diameter of the second section 22. The third section 23 has a diameter. The diameter of the third section 23 is smaller than the diameter of the second section 22 and may be the same as the diameter of the first section 21. The first section 21 comprises an end connected to the second end 12 of the needle 10. The second section 22 has an end connected to an opposite end of the first section 21. In the instant embodiment, the first section 21 of the suture thread 20 is tied to the connecting portion 121 of the second end 12 of the needle 10.

With the foregoing structure, when performing a closure process on a U-shaped surgical incision 30, the surgeon firstly inserts the needle 10 into the neighboring tissue of a first portion of the incision 30 and pulls the suture thread 20 that the third section 23 corresponds to the first portion of the incision 30, so as to perform a first suturing for first portion of the incision 30. The surgeon then pulls the suture thread 20 so to correspond the second section 22 to a second portion or an apex portion of the incision 30 to perform a second suturing. Further the surgeon pulls the suture thread 20 that the first section 21 corresponds to the third portion of the incision 30, so as to perform a third suturing for the third portion of the incision 30. A second-section-side portion of the third section 23 and a first-section-side portion of the second section 22 left unused and exposed outside the body of the patient may be removed later, for example, at the completion of the closure process.

The suture device in accordance with the present invention enables a physician or a surgeon to perform both the preliminary suturing and precise suturing required in a closure process and thus shortens operation time and provides great convenience.

What is claimed is:

1. A suture device comprising
a needle comprising
a point formed at a first end, and a suture thread connected to the needle and comprising
a first section comprising
a diameter, and
an end connected to a second end of the needle, and a second section comprising
a diameter different from the diameter of the first section,
and
an end connected to an opposite end of the first section.

2. The suture device as claimed in claim 1, wherein the diameter of the first section is smaller than the diameter of the second section.

3. The suture device as claimed in claim 2, wherein the needle further comprises
a connecting portion formed at the second end of the needle, and
wherein the first section of the suture thread is connected to the connecting portion of the needle.

4. The suture device as claimed in claim 2, wherein the needle is curved.

5. The suture device as claimed in claim 1, wherein the suture thread further comprises
a third section comprising
a diameter, and
an end connected to an opposite end of the second section.

6. The suture device as claimed in claim 5, wherein the diameter of the first section is smaller than the diameter of the second section, and
the diameter of the third section is smaller than the diameter of the second section.

7. The suture device as claimed in claim 6, wherein the needle further comprises
a connecting portion formed at the second end of the needle, and
wherein the first section of the suture thread is connected to the connecting portion of the needle.

8. The suture device as claimed in claim 6, wherein the needle is curved.

9. The suture device as claimed in claim 6, wherein the diameter of the third section is the same as the diameter of the first section.
